(19) United States
(12) Patent Application Publication
(10) Pub. No.: US 2026/0263137 A1
(43) Pub. Date: Sep. 10, 2026

(54) NON-INVASIVE NERVE AND VESSEL SEVERING DEVICE

(71) Applicant: Medtronic Ireland Manufacturing Unlimited Company, Dublin (IE)

(72) Inventor: Nicolas Coulombe, Anjou (CA)

(21) Appl. No.: 19/551,817

(22) Filed: Feb. 27, 2026

Related U.S. Application Data
(60) Provisional application No. 63/766,605, filed on Mar. 4, 2025.

(51) Int. Cl. A61B 18/02; A61B 18/00

(52) U.S. Cl. CPC A61B 18/0206; A61B 2018/0022; A61B 2018/00434; A61B 2018/00577; A61B 2018/00994; A61B 2018/0212; A61B 2018/0262

(57) ABSTRACT

A catheter includes an elongate body that defines longitudinal axis and includes a proximal end and a distal end. The catheter also includes a shaft disposed in the elongate body, a fluid delivery channel for delivering a coolant, and a first expandable structure coupled to the distal end of the elongate body and the shaft. The first expandable structure defines a first interior chamber configured to receive the coolant. The catheter also includes a second expandable structure coupled to the distal end of the elongate body and the shaft adjacent the first expandable structure. The second expandable structure defines a second interior chamber. The catheter also includes a transducer mounted to the shaft within the second interior chamber, and the transducer is configured to emit vibrational energy within second interior chamber. The second interior chamber contains a fluid, and the vibrational energy propagates through the fluid to a treatment region.

### CROSS-REFERENCE TO RELATED APPLICATIONS

This application claims priority to U.S. Provisional Patent Application No. 63/766,605, filed on Mar. 4, 2025, the entire contents of which are incorporated herein by reference.

### BACKGROUND

Catheter based devices are employed in various medical and surgical applications as non-invasive, precise treatment tools for targeting localized issues that are otherwise inaccessible. Catheters may be easily inserted and navigated through the blood vessels and arteries, allowing non-invasive access to areas with relatively little trauma. Catheter-based systems have been developed for implementation in tissue ablation for treatment of cardiac arrhythmias, such as atrial fibrillation, supra ventricular tachycardia, ventricular fibrillation, and the like. One such system involves cryoablation catheters, which use cryogens (fluids with low operating temperatures) to selectively freeze, or “cold-treat,” targeted tissues within the body. Cryoablation catheters typically include an expandable element, such as a balloon, at the distal end that contacts the selected tissue.

There is interest in utilizing cryoablation for denervation treatment of intractable diseases, such as renal denervation for hypertension, or bronchial denervation for chronic obstructive pulmonary disease, etc. However, ablating renal or bronchial tissue with cryo-energy alone may not be enough to permanently eliminate nerve regrowth in the targeted tissue. Among other things, a catheter that provides both cryo-energy and vibrational energy may provide certain advantages. In one example, and as is explained in more detail below, vibrational energy is applied after cryo-energy is applied to create cracks is frozen tissue, and as a consequence, physically sever the frozen tissue.

One example provides, among other things, a catheter including an elongate body defining a longitudinal axis, and the elongate body includes a proximal end and a distal end. The catheter also includes a shaft disposed in the elongate body, a fluid delivery channel for delivering a coolant, and a first expandable structure coupled to the distal end of the elongate body and the shaft. The first expandable structure defines a first interior chamber configured to receive the coolant. The catheter also includes a second expandable structure coupled to the distal end of the elongate body and the shaft adjacent the first expandable structure. The second expandable structure defines a second interior chamber. The catheter also includes a transducer mounted to the shaft within the second interior chamber of the second expandable structure, and the transducer is configured to emit vibrational energy within second interior chamber. The second interior chamber contains a fluid, and the vibrational energy propagates through the fluid to a treatment region.

Another example provides, among other things, a method of cryoablating a treatment region using a catheter that includes a fluid delivery channel for delivering a coolant, a first expandable structure having a first interior chamber configured to receive the coolant, and a second expandable structure having a second interior chamber with a fluid. The catheter also includes a transducer configured to emit vibrational energy within the second interior chamber and through the fluid. The method includes inserting the catheter into a vascular site of a patient, expanding the first expandable structure to contact a treatment region within the patient, and delivering coolant through the fluid delivery channel to the first interior chamber of the first expandable structure. The method also includes cryoablating the treatment region with the first expandable structure, expanding the second expandable structure, and emitting vibrational energy that propagates through the fluid in the second interior chamber and to the treatment region or adjacent to the treatment region.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments, examples, aspects, and features that include the claimed subject matter, and explain various principles and advantages.

FIG. 1 is a perspective view of an example of a medical device.

FIG. 2 is a cross-sectional view of a distal portion of the medical device of FIG. 1.

FIG. 3 is a schematic view of the distal portion of the medical device of FIG. 1 within a patient’s vasculature.

FIG. 4 is a zoomed-in schematic view of the medical device of FIG. 3 illustrating a treatment region within the patient.

FIG. 5 is a schematic view of another example of a medical device.

FIG. 6 is a schematic view of another example of a medical device.

FIG. 7 is a zoomed-in schematic view of the medical device of FIG. 6 illustrating a treatment region within the patient.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments, examples, aspects, and features.

The apparatus and method components have been represented where appropriate by conventional symbols in the drawings, showing only those specific details that are pertinent to understanding the embodiments, examples, aspects, and features described and illustrated so as not to obscure the disclosure with details that will be readily apparent to those of ordinary skill in the art having the benefit of the description herein.

### DETAILED DESCRIPTION

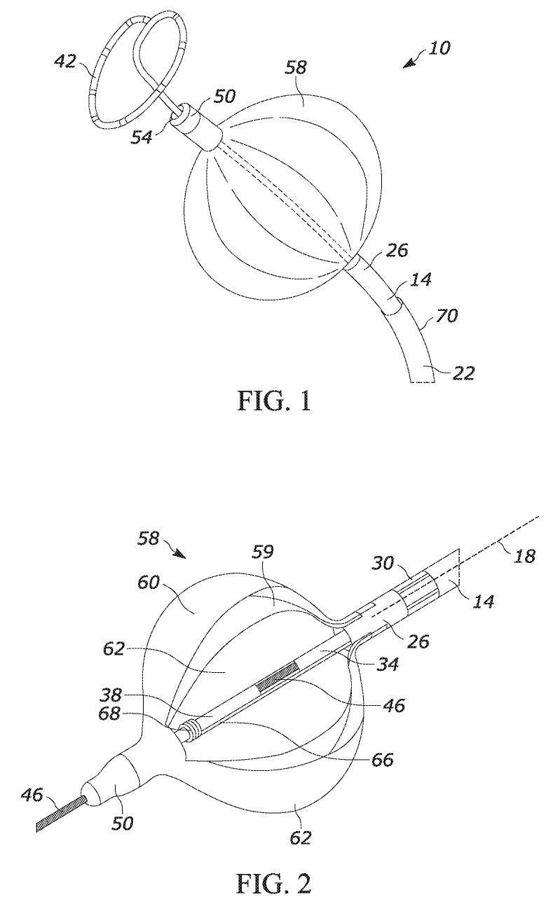

FIG. 1 illustrates a medical device 10 that may be coupled to a generation unit or operating console (not shown). The medical device 10 may generally include one or more diagnostic or treatment areas for energetic, therapeutic, and/or investigatory interaction with a patient.

In the example shown in FIGS. 1 and 2, the medical device 10 (e.g., a catheter) includes an elongate body 14 for insertion within a patient’s vasculature and/or proximate to a tissue region for diagnosis, treatment, and/or mapping. The elongate body 14 defines a longitudinal axis 18 (FIG. 2) and includes a proximal end 22 and a distal end 26 opposite the proximal end 22. The elongate body 14 also defines a lumen 30 for receiving an elongate shaft 34 that is partially disposed within a portion of the elongate body 14. The elongate shaft 34 can selectively extend from the elongate body 14 and is movable with respect to the elongate body 14 along the longitudinal axis 18 or other directions transverse to the axis 18. The elongate shaft 34 defines a lumen 38 therein for the introduction and passage of a mapping electrode assembly 42 and/or a guide wire 46 (FIG. 2). The mapping electrode assembly 42 is configured to sense electric signals (e.g., electrical activity of an organ) from the treatment region and transmit the signals (or processed versions of them) to a control unit to obtain a baseline electrophysiological map of electrical activity in selective tissue in the treatment region. The guide wire 46 is configured to stabilize the elongate body 14 as the device 10 moves through the patient’s vasculature and to the treatment region. The elongate shaft 34 also includes a distal tip 50 having an opening 54 to allow the mapping electrode assembly 46 and/or the guide wire 42 to extend therethrough.

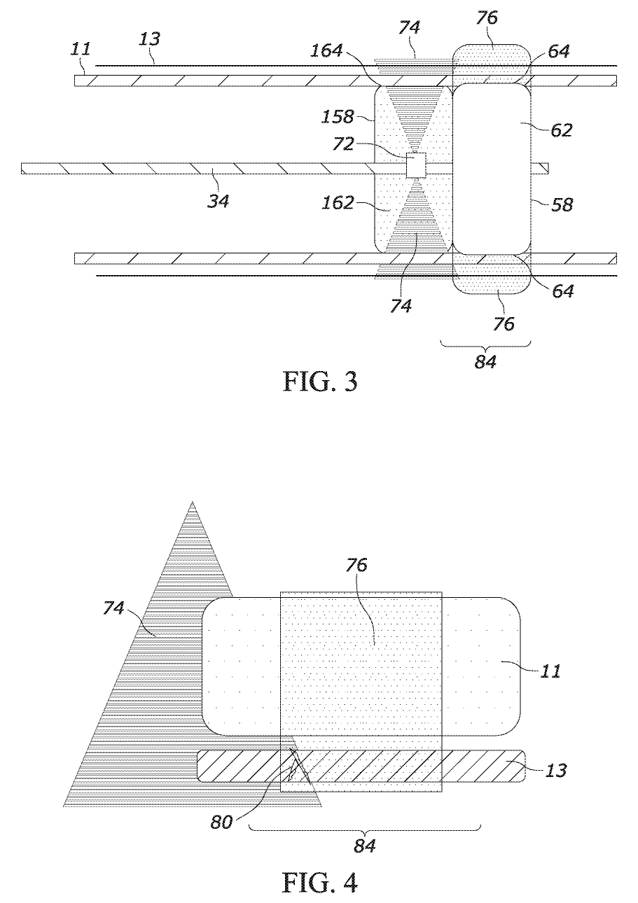

With continued reference to the example shown in FIGS. 1 and 2, the medical device 10 also includes one or more expandable structures 58, 158 (FIG. 3), such as a balloon, located on the distal end 26 of the elongate body 18 and coupled to the elongate shaft 34 and the distal tip 50. The expandable structure 58 defines an internal chamber 62. In some examples, the expandable structure 54 includes a first layer 59 and a second layer 60 that is a back-up layer in case the first layer 59 is damaged. The medical device 10 also includes a fluid delivery conduit 66 coupled to the elongate shaft 34 for delivering a fluid (e.g., a refrigerant, or a coolant, such as nitrous oxide, carbon dioxide, nitrogen, or argon) to the internal chamber 62 of the expandable structure 54 from a fluid supply in the operating console. In some examples, the fluid delivery conduit 66 is wrapped around the elongate shaft 34 in a coil 68 having a plurality of openings for delivering fluid to the internal chamber 62. In other examples, the fluid delivery conduit 58 is coupled to the elongate shaft 34.

With reference to FIGS. 1-3, the medical device 10 also includes a transducer 72. In one instance (FIG. 3), the transducer is coupled to the elongate shaft 34 and positioned within the expandable structure 158. Like the expandable structure 58, the expandable structure 158 is located on the distal end 26 of the elongate body 18 and coupled to the elongate shaft 34. The expandable structure 158 also defines an internal chamber 162 for housing the transducer 72. However, unlike the expandable structure 58, the internal chamber 162 of the expandable structure 158 does not receive refrigerant/coolant from the fluid delivery conduit 66. Instead, the fluid delivery conduit 66 is configured to deliver a fluid to the internal chamber 162 from the fluid supply in the operating console. In some examples, the fluid is a predominantly non-compressible fluid, such as distilled water, saline, radio-opaque contrast agent or other non-compressible fluids known in the art. In other examples, the fluid is a compressible fluid, such as air, or other such compressible fluids known in the art. The transducer 72 is configured to emit vibrational energy 74 (e.g., sound waves) that propagates through the fluid within the internal chamber 162 of the expandable structure 158. The fluid acts as a wave guide for guiding the vibrational energy 74 to a treatment region 84. In some examples, the transducer 72 frequency can be tuned so that the maximum vibrational energy 74 is focused at a particular depth of the fluid within the internal chamber 162 of the expandable structure 158. In other examples, the transducer 72 frequency can be focused at a particular depth outside of the internal chamber 162 of the expandable structure 158 and within a defined area in the treatment region 84. In yet other examples, the transducer 72 can be a remote transducer located externally from the expandable structure 158 for emitting vibrational energy 74. In yet other examples, the transducer 72 is a piezo electric transducer formed from ceramic components and configured to emit vibrational energy in the form of ultrasonic sound waves in a frequency range between 0.5-4.0 MHz.

With continued reference to the examples shown in FIGS. 1-3, the medical device 10 also includes a sheath 70 (FIG. 1). In some instances, the sheath 70 is selectively movable relative to the one or more expandable structures 58, 158 between a first configuration and a second configuration. In the first configuration, the one or more expandable structures 58, 158 are located outside the sheath 70 in an expanded configuration where each of the expandable structures 58, 158 forms a substantially spherical shape defining an outer circumferential profile 64, 164 for contacting the treatment region 84. In some examples, the outer circumferential profile 164 of the expandable structure 158 does not contact the treatment region 84. When the medical device 10 is positioned in the first configuration, cryoablation can be performed with the expandable structure 58, apply vibrational energy 74 via the expandable structure 158, and/or perform mapping with the mapping electrode assembly 42. In the second configuration, the sheath 70 extends over the elongate body 14 and the expandable structures 58, 158 are retracted and housed within the sheath 70 so the device 10 is more easily maneuverable within the patient’s vasculature.

Use of the device 10 to cryoablate the treatment region 84 may be understood by referring to FIGS. 1-4. In one example, the user inserts the device 10 adjacent to the treatment region 84 within the vasculature of the patient when the device 10 is in the second configuration. The treatment region 84 includes a targeted vessel wall 11 and/or nerve 13 adjacent the vessel wall 13. The user moves the device 10 into the first configuration and can either map the treatment region 84 via the mapping electrode assembly 42, or begin expanding the one or more expandable structures 58, 158. Once the expandable structures 58, 158 are expanded, the user then positions the expandable structure 58 such that the outer circumferential profile 64 of the expandable structure 58 contacts the treatment region 84. Coolant is delivered via the fluid delivery conduit 66 to the expandable structure 58, which fills the internal chamber 62 with coolant, thereby causing an ice mass 76 to form on the treatment region 84. This ice mass 76 ablates both the targeted vessel 13 and nerve 11. Fluid is delivered via the fluid delivery conduit 66 to the expandable structure 158, and the transducer 72 is activated, which emits vibrational energy 74. The vibrational energy 74 propagates through the fluid and onto or adjacent to the treatment region 84 to cause movement within the ice mass 76 and break apart the ice mass 76 surrounding the frozen nerve 13. When the ice mass 76 begins to break apart, the ice mass 76 fractures, causing tears, or cracks 80 to form in the nerve 13 to physically disrupt and sever the nerve 13. Once the nerve 13 is severed, the user retracts the one or more expandable structures 58, 158 within the sheath 70 to move the device 10 to the second configuration for removal from the patient.

In some examples, the transducer 72 is a piezo electric transducer. In other examples, the piezo electric transducer is configured to emit ultrasonic sound waves into the ice mass 76 to break apart the ice mass 76. When the ice mass 76 is formed, air bubbles can naturally form within the ice mass 76, and ultrasonic sound emitted from the piezo electric transducer can facilitate cavitation within the ice mass 76, thereby causing the ice mass 76 to fracture. In other examples, the frequency of the vibrational energy emitted from the piezo electric transducer can be calibrated according to a resonant frequency of the ice mass 76, which can vary depending on the volume of the ice mass 76. In yet other examples, the transducer 72 is positioned on the elongate shaft 34 according to the distance the emitted vibrational energy 74 needs to travel in order to contact the point within the ice mass 76 that encapsulates the targeted nerves 13. In yet other examples, the transducer 72 can be coupled to the shaft 34 with adhesives (e.g., a glue)or be thermally fused to the shaft 34

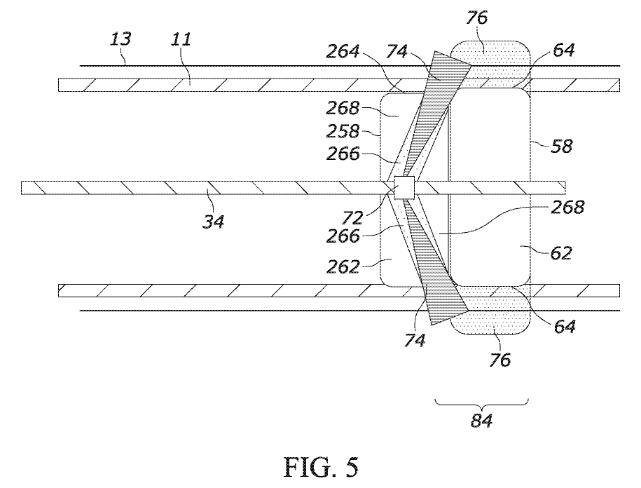

FIG. 5 illustrates an alternative example of an expandable structure 258 for use with the medical device 10. In this example, the expandable structure 258 is located on the distal end 26 of the elongate body 18 and coupled to the elongate shaft 34 adjacent the expandable structure 58. The expandable structure 258 defines an outer circumferential profile 264 for contacting the treatment region 84 when the expandable structure 258 is in the first configuration. The expandable structure 258 also defines an interior chamber made up of a first chamber 266 and a second chamber 268 surrounding the first chamber 266. The first chamber 266 is configured to house the transducer 72 that is mounted to the elongate shaft 34 within the first chamber 266. The first chamber 266 is also configured to receive a first predominantly non-compressible fluid, such as distilled water, from the fluid delivery conduit 66. The second chamber 268 is configured to be filled with a second compressible fluid that is different than the first non-compressible fluid, such as air.

In operation, when the transducer 72 emits the vibrational energy 74, and the outer circumferential profile 264 of the expandable structure 258 contacts or is adjacent to the treatment region 84, the first chamber 266 acts as a wave guide allowing the vibrational energy 74 to propagate through the first non-compressible fluid onto or adjacent to the treatment region 84. The second chamber 268 does not allow the vibrational energy 74 to propagate through the second compressible fluid, thereby acting as an insulator for the surrounding areas of the treatment region 84 that are in contact with the portions of the second chamber 268 that abut the outer circumferential profile 264. In some examples, the size of the second chamber 268 can be altered to expand or shrink the size of the first chamber 266, thereby altering amount of the treatment region 84 receiving the vibrational energy 74 from the transducer 72.

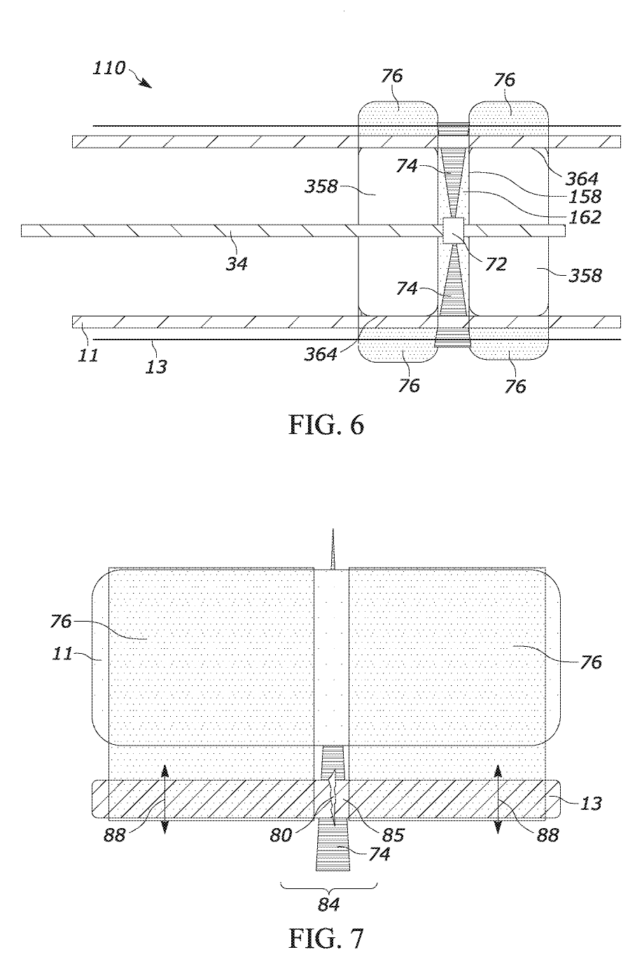

FIG. 6 and 7 illustrate another example of a medical device 110 that is similar to the medical device 10 with differences explained below. The medical device 110 includes a plurality of expandable structures 358 (e.g., a balloon) each mounted to the distal end 26 of the elongate body 18 and coupled to the shaft 34. Each of the plurality of expandable structures 358 define an outer circumferential profile 364 for contacting a portion of the treatment region 84 (FIG. 7). During operation of the device 110, each of the plurality of expandable structures 358 is configured to receive refrigerant/coolant from the fluid delivery conduit 66 (FIG. 2) and freeze a portion of the treatment region 84 to create ice masses 76 that encompass the targeted vessels 11 and nerves 13. The medical device 110 also includes another expandable structure 158 (e.g., a balloon) coupled to the shaft 34 between the plurality of expandable structures 358, and a transducer 72 coupled to the shaft 34 and located within the expandable structure 158. The expandable structure 158 defines an internal chamber 162 for both housing the transducer 72 and a fluid. In some examples, the fluid is a predominantly non-compressible fluid, such as distilled water, saline, radio-opaque contrast agent, or other non-compressible fluids known in the art. In other examples, the fluid is a compressible fluid, such as air, or other compressible fluids known in the art. The transducer 72 is configured to emit vibrational energy 74 (e.g., sound) within the expandable structure 158 and through the fluid. The fluid acts as a wave guide for allowing vibrational energy 74 to propagate through the fluid and be guided to the treatment region 84.

To sever a targeted nerve 11 in the treatment region 84, the plurality of expandable structures 358 are utilized to form respective ice masses 76 on respective portions of the treatment region 84 that encapsulate the targeted nerve 13. Since the plurality of expandable structures 358 are separated by the expandable structure 158, there is an unfrozen portion 85 (FIG. 7) of the targeted nerve 13 that is located between the two respective ice masses 76. The transducer 72 is activated (for example, manually or automatically) to emit vibrational energy 74 through the fluid and into or adjacent the unfrozen portion 85 of the treatment region 84. As the vibrational energy 74 propagates through the fluid and into the treatment region 84, the energy 74 causes respective ice masses 76 to move relative to the targeted nerve 13, thereby creating a shear force on the unfrozen portion 85 of the treatment region 84 in a direction 88 transverse to the targeted nerves 13. This shear force causes tears, or cracks 80 to form in the nerves 13 within the unfrozen portion 85 between the respective ice masses 76, and eventually causes the nerves 13 to sever and become permanently damaged. In some examples, multiple freeze/thaw cycles of the ice masses 76 with variation of the frequency of the vibrational energy 74 can further create irreversible damage to the targeted nerves 13. In other examples, the medical device 110 can be utilized with different target structures in the treatment region 84 around which hyperactive autonomic nerve networks interface, such as bronchi, other vessel types, and bones.

In some examples, each of the medical devices 10, 110 can include a plurality of electrodes coupled to the shaft and disposed within an expandable structure to create the vibrational energy instead of the transducer. In such examples, the expandable structure includes a medium having a high density for containing the plurality of electrodes, and the plurality of electrodes create an arc therebetween to create a shockwave that propagates through the medium and onto the treatment region. This shockwave fractures the ice masses to cause death of the targeted vessels or nerves encapsulated by the ice masses. Also, in such examples, the energy supply to the plurality of electrodes can include a high voltage, low current energy source, such as pulsed field energy.

In the foregoing specification, specific examples have been described. However, one of ordinary skill in the art appreciates that various modifications and changes can be made without departing from the scope of the claimed subject matter. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises …a,” “has …a,” “includes …a,” or “contains …a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially,” “essentially,” “approximately,” “about,” or any other version thereof, are defined as being close to as understood by one of ordinary skill in the art, and in one non-limiting example the term is defined to be within 10%, in another example within 5%, in another example within 1% and in another example within 0.5%. The term “coupled” as used herein is defined as connected, although not necessarily directly and not necessarily mechanically. A device or structure that is “configured” in a certain way is configured in at least that way but may also be configured in ways that are not listed.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separately claimed subject matter.

What is claimed is:

1. A catheter comprising:
an elongate body defining a longitudinal axis, the elongate body including a proximal end and a distal end;
a shaft disposed in the elongate body;
a fluid delivery channel for delivering a coolant;
a first expandable structure coupled to the distal end of the elongate body and the shaft, the first expandable structure defining a first interior chamber configured to receive the coolant;
a second expandable structure coupled to the distal end of the elongate body and the shaft adjacent the first expandable structure defining a second interior chamber; and
a transducer mounted to the shaft within the second interior chamber of the second expandable structure, the transducer configured to emit vibrational energy within second interior chamber;
wherein the second interior chamber contains a fluid and the vibrational energy propagates through the fluid to a treatment region.

2. The catheter of claim 1, wherein the fluid is a first non-compressible fluid, and wherein the second interior chamber contains a plurality of separate chambers within the second interior chamber with one of the plurality of separate chambers filled with the first non-compressible fluid and the other one of the plurality of separate chambers is filled with a second compressible fluid different from the first non-compressible fluid.

3. The catheter of claim 2, wherein the second compressible fluid does not allow for the propagation of the vibrational energy.

4. The catheter of claim 1, wherein the first expandable structure is configured to freeze the treatment region, and the transducer emits vibrational energy through the fluid to the treatment region or adjacent to the treatment region.

5. The catheter of claim 4, wherein the first expandable structure is substantially spherical in shape and defines an outer circumferential treatment area for contacting the treatment region.

6. The catheter of claim 5, wherein, when the first expandable structure freezes the treatment region, the first expandable structure forms a frozen mass on the treatment region that fractures in response to receiving the vibrational energy from the transducer.

7. The catheter of claim 1, wherein the fluid delivery channel is coupled to the shaft.

8. The catheter of claim 1, wherein the shaft defines a lumen though which a sensor is movable therethrough.

9. The catheter of claim 1, wherein the transducer is a piezo electric transducer that operates within a frequency range between 0.5–4.0 MHz.

10. The catheter of claim 1, wherein the first expandable structure is a plurality of first expandable structures positioned on opposite sides of the second expandable structure that are each configured to freeze a portion of the treatment region.

11. The catheter of claim 10, wherein, each of the plurality of first expandable structures forms a frozen mass on their respective portions of the treatment region, and an unfrozen area between the respective portions of the treatment region fractures in response to receiving the vibrational energy from the transducer.

12. A method of cryoablating a treatment region using a catheter, the catheter includes a fluid delivery channel for delivering a coolant, a first expandable structure having a first interior chamber configured to receive the coolant, a second expandable structure having a second interior chamber with a fluid, and a transducer configured to emit vibrational energy within the second interior chamber and through the fluid, the method comprising:
inserting the catheter into a vascular site of a patient;
expanding the first expandable structure to contact a treatment region within the patient;
delivering coolant through the fluid delivery channel to the first interior chamber of the first expandable structure;
cryoablating the treatment region with the first expandable structure;
expanding the second expandable structure; and
emitting vibrational energy that propagates through the fluid in the second interior chamber and to the treatment region or adjacent to the treatment region.

13. The method of claim 12, wherein the fluid is a first non-compressible fluid, and wherein the second interior chamber contains a plurality of separate chambers within the second interior chamber with one of the plurality of chambers filled the first non-compressible fluid and the other one of the plurality of separate chambers is filled with a second compressible fluid different from the first non-compressible fluid.

14. The method of claim 13, wherein the second compressible fluid does not allow for the propagation of the vibrational energy.

15. The method of claim 12, wherein cryoablating the treatment region with the first expandable structure includes freezing the treatment region to form a frozen mass on the treatment region.

16. The method of claim 15, further comprising fracturing the frozen mass with the vibrational energy from the transducer.

17. The method of claim 12, wherein the transducer is a piezo electric transducer that operates within a frequency range between 0.5–4.0 MHz.

18. The method of claim 12, wherein the first expandable structure is a plurality of first expandable structures positioned on opposite sides of the second expandable structure that are each configured to freeze a portion of the treatment region.

19. The method of claim 18, wherein, cryoablating the treatment region with the first expandable structure includes forming a frozen mass on respective portions of the treatment region corresponding to each of the first expandable structures and forming an unfrozen area between the respective portions of the treatment region.

20. The method of claim 19, further comprising fracturing the unfrozen area between the respective portions of the treatment area with the vibrational energy from the transducer.
